(19) United States
(12) Patent Application Publication
(10) Pub. No.: US 2025/0295371 A1
(43) Pub. Date: Sep. 25, 2025

(54) EVALUATION OF CHARACTERIZATION DATA OF AN X-RAY DETECTOR

(71) Applicant: Siemens Healthineers AG, Forchheim (DE)

(72) Inventor: Florian WOLZ, Erlangen (DE)

(73) Assignee: Siemens Healthineers AG, Forchheim (DE)

(21) Appl. No.: 19/085,169

(22) Filed: Mar. 20, 2025

(30) Foreign Application Priority Data
Mar. 22, 2024 (DE) 10 2024 202 766.9

(51) Int. Cl. A61B 6/58; A61B 6/03

(52) U.S. Cl. CPC A61B 6/583; A61B 6/03

(57) ABSTRACT

One or more example embodiments relates to a computer-implemented method for supporting the evaluation of characterization data of an X-ray detector for an X-ray imaging system, in particular for a computed tomography system, with a plurality of detector modules, wherein the method comprises the following steps: receiving characterization data for the detector modules of the X-ray detector, wherein at least part of the characterization data is based on measurement data of the detector modules recorded without an examination object; applying a trained algorithm to the characterization data, wherein the output generated is synthetic image data simulating image data of an X-ray imaging system, in particular a computed tomography system, recorded with the X-ray detector; providing the synthetic image data.

### CROSS-REFERENCE TO RELATED APPLICATION(S)

The present application claims priority under 35 U.S.C. § 119 to German Patent Application No. 10 2024 202 766.9, filed Mar. 22, 2024, the entire contents of which is incorporated herein by reference.

### FIELD

Independent of the grammatical term usage, individuals with male, female or other gender identities are included within the term.

One or more example embodiments relates to a computer-implemented method for supporting the evaluation of characterization data of an X-ray detector for an X-ray imaging system, a method for quality control in the manufacture of an X-ray detector for an X-ray imaging system, a computer program product or storage medium, a system and a method for training a trainable algorithm.

### RELATED ART

X-ray detectors such as computed tomography detectors (CT detectors) typically consist of a plurality of subcomponents, in particular detector modules. Herein, the image quality that can be achieved with a respective X-ray detector depends upon the detector modules installed. Herein, it has been shown that, in addition to the features of the individual detector modules, other factors, such as relating to a combination of detector modules, can also play a role in image quality.

It is therefore important to perform image tests on images of X-ray detectors (hereinafter also referred to as “detectors” for short) in order, for example, to detect regularly occurring detector-related artifacts or other errors. However, a plurality of very time-consuming steps is necessary to reach the image testing stage. It can take a relatively long time to run through the corresponding test chains for testing a detector. Hence, testing image quality involves a not insignificant amount of time and money. Furthermore, a change to the detector, for example, by replacing one or more modules, can entail the risk that the image quality will subsequently deteriorate or no longer meet the desired or required quality criteria.

For this reason, one approach can be to make a statement about the expected image quality, for example, based on a few scans. For example, characterization measurement data can be recorded. This characterization measurement data can, for example, be analyzed with the aid of defined limit values in order to make a statement about whether image artifacts are expected. The limit values can, for example, be defined by experts based on empirical values. Hence, in particular, estimations of image quality can be made without looking at actual image data or even having created it.

### SUMMARY

However, in many cases, such an analysis is qualitative rather than quantitative in nature. The precision or reliability regarding actual image quality is therefore generally limited. This analysis therefore can overlook detectors with insufficient quality or there is a possibility of detectors with a quality that would actually be sufficiently good being rejected.

One or more example embodiments provides a way of enabling a more reliable early-stage estimation of an image quality of an X-ray detector.

### BRIEF DESCRIPTION OF THE DRAWINGS

The following describes embodiments with reference to the attached figures.

FIG. 1 shows a flowchart for a computer-implemented method for supporting the evaluation of characterization data of an X-ray detector for an X-ray imaging system according to one embodiment of the invention,

FIG. 2 shows a flowchart for a computer-implemented method for training a trainable algorithm according to one embodiment of the invention,

FIG. 3 shows a schematic diagram of the structure of a trained or trainable algorithm according to one embodiment of the invention,

FIG. 4 shows schematically the mode of operation of the diffusion model shown in FIG. 4,

FIG. 5 shows a flowchart for a computer-implemented method for training a trainable algorithm based on a diffusion model according to one embodiment of the invention,

FIG. 6 shows a schematic structure of a system according to one embodiment of the invention, and

FIG. 7 shows a method for quality control in the manufacture of an X-ray detector for an X-ray imaging system with a plurality of detector modules according to one embodiment of the invention.

### DETAILED DESCRIPTION

The following describes solutions according to example embodiments of the invention with reference to the claimed systems, products and methods. Furthermore, the following describes the solutions according to example embodiments of the invention with reference to the claimed systems, products and methods for supporting the evaluation of characterization data or for quality control in the manufacture of an X-ray detector as well as with reference to the systems, products and methods for providing a trained algorithm. Features, advantages or alternative embodiments described herein with respect to one aspect according to the invention can in each case be transferred analogously to the other aspects and vice versa. In other words, claims and embodiments for systems and/or products according to example embodiments of the invention can be improved by features described or claimed in the context of the respective methods. Functional features of a method can be implemented by physical entities of the system and/or product. Features, advantages or alternative embodiments. Claims and embodiments for providing a trained algorithm can be improved by features described or claimed in the context of the systems, products and methods for supporting the evaluation of characterization data or for quality control in the manufacture of an X-ray detector. In particular, data sets used for systems, products and methods for supporting the evaluation of characterization data or for quality control in the manufacture of an X-ray detector can have the same properties and features as the corresponding data sets used in the systems, products and methods for providing a trained algorithm. Trained algorithms provided by the corresponding methods, products and systems can be used in the systems, products and methods for supporting the evaluation of characterization data or for quality control in the manufacture of an X-ray detector.

According to one or more example embodiments of the invention, a computer-implemented method is provided for supporting the evaluation of characterization data of an X-ray detector for an X-ray imaging system, in particular for a computed tomography system, with a plurality of detector modules. The method comprises the following steps:
receiving characterization data of the detector modules of the X-ray detector, wherein at least part of the characterization data is based on measurement data of the detector modules recorded without an examination object;
applying a trained algorithm to the characterization data, wherein the output generated is synthetic image data simulating image data of an X-ray imaging system, in particular a computed tomography system, recorded with the X-ray detector;
providing the synthetic image data.

Advantageously, the method according to one or more example embodiments of the invention can be used to provide image data at a point in time during the setting up of the X-ray detector, which is before the point in time at which image data is usually available via a direct measurement. In particular, the synthetic image data can generally be obtained much more quickly than image data obtained by real image measurements. The synthetic image data can be a particularly reliable estimation of actual image data. Hence, the synthetic image data can provide a more direct insight into how the image quality of image data recorded later will turn out. Hence, information about image quality can be obtained at an early stage with a high degree of reliability. For example, artifacts, such as, for example, ring artifacts or streak artifacts, can be easily identified in the synthetic image data. Hence, if errors are detected, it is possible to respond at an early stage and make corrections. Hence, in particular, this enables a better evaluation of the characterization data. Evaluation of the characterization data can, for example, comprise an assessment and/or evaluation of the characterization data. In the context of example embodiments, an evaluation of characterization data can in particular be an evaluation of the quality of the X-ray detector or the quality of the image data that can be recorded with the X-ray detector. In particular, the method according to one or more example embodiments of the invention can use the synthetic image data to provide an indicator of the effects of changes to the X-ray detector, in particular with regard to the detector modules, at an early stage in a manufacturing process and/or in a maintenance process. On the one hand, this can speed up a production process. However, the method can also, for example, be used to advantage when replacing detector modules (for example, during service calls) and when planning a replacement.

The term “characterization data” is to be understood broadly in the context of the present invention. Characterization data is generally data that can be used to characterize the X-ray detector and/or the individual detector modules of the X-ray detector. Characterization data can be based on measurement data of the detector modules. The characterization data can, for example, be raw data that in particular corresponds to the directly recorded measurement data of the detector modules. The characterization data can comprise further processed measurement data. The characterization data can comprise data that was not recorded directly by the detector modules. For example, the characterization data can comprise a temperature of the detector modules captured by a temperature sensor and/or temperature in the environment of the detector modules. Preferably, characterization data is data that was recorded while the X-ray imaging system was not yet fully set up and/or while no image data of an examination object was recorded with the X-ray detector. The examination object can also be referred to as a measurement object. The examination object can, for example, be a human or an animal or a part of a human or an animal. An examination object can, for example, be an object, such as an item of baggage during a baggage check. Image data can, for example, be images, in particular of an examination object. Image data can be raw data, in particular of an examination object, which can be used in an image reconstruction process to create images, in particular of the examination object.

The term “X-ray detector” is to be understood broadly in the context of the present invention. Generally, an X-ray detector refers to a detector for capturing X-rays. The X-ray detector is provided for an X-ray imaging system. In particular, the X-ray detector is provided for a computed tomography system. The X-ray detector comprises a plurality of detector modules. The X-ray detector can be embodied to convert X-rays into electrical signals using the detector modules. The plurality of detector modules can preferably be embodied as a matrix-like arrangement. For example, 10-200, preferably 20-100, detector modules can be provided for an X-ray detector. The detector modules of the X-ray detector can be detector modules of the same class or the same design. However, it has been shown that, in reality, even detector modules of the same class often have at least slight differences. Such differences can lead to artifacts in imaging. It is not always easy to predict whether and to what extent such artifacts will occur. It has been shown that, on the one hand, in reality, slightly different detector modules can lead to artifacts, for example, ring artifacts, and that, on the other hand, even faulty detector modules can possibly still lead to good image data. Advantageously, the synthetic image data of the method according to one or more example embodiments of the invention can be used to make better predictions about the occurrence of artifacts.

The method comprises a step of receiving the characterization data of the detector modules of the X-ray detector. The characterization data can, for example, be received via an interface. The characterization data can, for example, be retrieved from a database. The characterization data can be retrieved locally and/or retrieved from a network and/or a remote connection, for example, via the internet. The characterization data can, for example, be input by a user into corresponding processing software designed to perform the method according to one or more example embodiments of the invention. Optionally, the method according to one or more example embodiments of the invention can be performed on a computer unit of an X-ray imaging system. For example, the characterization data can be generated by the X-ray imaging system itself and forwarded to the computer unit. The characterization data can have been created while the detector modules of the X-ray detector were already installed together in the X-ray detector. Optionally, the characterization data for the detector modules can have been created in whole or in part while the detector modules were not installed together in the X-ray detector and/or while the detector modules were installed in a configuration other than the arrangement to which they are now assigned. Characterization data is in particular data that is suitable for characterizing, individually and/or in their entirety, at least some of the properties of the detector modules and/or the X-ray detector. At least part of the characterization data is based on measurement data of the detector modules recorded without an examination object. Preferably, all characterization data can be based on data that was not recorded in a method corresponding to the normal intended operation of the X-ray detector with an examination object. Measurement data of the detector modules recorded without an examination object, can, for example, be or comprise air shot data of the detector modules. Measurement data of the detector modules that was recorded without an examination object can, for example, be data of the detector modules that was recorded under X-ray irradiation. The characterization data can, for example, be provided as a vector or matrix. For example, different detector modules can be encoded by numbers.

A trained algorithm is applied to the characterization data. The trained algorithm can in particular be based on machine learning. The term “trained algorithm” can in particular comprise the various aspects of machine learning. The trained algorithm can also be referred to as a trained function. In particular, the algorithm is able to adapt to new circumstances based on training data and to recognize and extrapolate patterns. Generally, parameters of the algorithm can be adapted by training. The training can, for example, comprise supervised learning, semi-supervised learning, active learning, self-supervised learning, unsupervised learning and/or reinforcement learning. In particular, the parameters of the algorithm can be adapted iteratively through a plurality of training steps. In particular, a specific loss function can be optimized, in particular minimized, during training. For example, the trained algorithm can be an artificial neural network, a support vector machine, a decision tree, in particular a random forest, and/or a Bayesian network. Additionally or alternatively, the algorithm can be based on a k-means algorithm, a Q-learning algorithm, an evolutionary algorithm, a Monte-Carlo tree search and/or on association analysis. In particular, a backpropagation algorithm (error feedback algorithm) can be used in the context of training a neural network. A neural network can in particular be a deep neural network (DNN), convolutional neural network (CNN) or convolutional deep neural network. The trained algorithm preferably comprises a generative model. The trained algorithm is configured or trained to output synthetic image data.

The term “synthetic image data” is to be understood broadly in the context of the present invention. Image data generally describes data from which at least one visual medium, in particular two-dimensional or three-dimensional images, can be generated. Synthetic image data is in particular image data that is completely or partially artificially generated. In particular, synthetic image data can have been generated by an algorithm. In the context of the present invention, the synthetic image data corresponds to image data of an X-ray imaging system recorded with the X-ray detector. In particular, the synthetic image data can correspond to data of a computed tomography system that was recorded with the X-ray detector. The synthetic image data can be obtained significantly more quickly with the method according to one or more example embodiments of the invention than real image data can be obtained with a corresponding measurement. In the context of the present invention, it was recognized that characterization data that can be generated at an early stage in the manufacturing process of the X-ray detector can be decisive for artifacts that occur in real images that are generated later. Advantageously, example embodiments make use of this circumstance by generating the synthetic image data based on the characterization data.

The synthetic image data is provided. The ‘provision’ is generally to be understood broadly in the context of the present invention. For example, output can be provided via an output medium, in particular for a user. The output medium can, for example, be a screen, a projector, or a printer. The provision can, for example, be an output for further processing, for example forwarding to another program and/or an external device. The provision can, for example, comprise storage on an external or internal data carrier.

According to one embodiment, the characterization data can be assigned to an arrangement of the detector modules in the X-ray detector. For example, characterization data can be provided for each detector module, wherein the characterization data of each detector module is assigned to a position in the arrangement of the detector modules. The assignment can, for example, be provided via coordinate data. The assignment can, for example, be provided via a sequence of the characterization data. For example, the first characterization data in a set of characterization data can be assigned to a detector module in a first position in the X-ray detector and the last characterization data in the set of characterization data can be assigned to a detector module in a last position in the X-ray detector. For example, the first position can be at the top left of the X-ray detector and/or the last position can be at the bottom right of the X-ray detector. Herein, the characterization data can, for example, be provided as a vector or matrix. For example, different detector modules can be encoded by numbers.

According to one embodiment, the characterization data comprises one or more of the following:
a response of detector pixels of the detector modules to radiation without an examination object,
temporal noise behavior of individual detector pixels of the detector modules,
signal instabilities of the detector modules induced by incoming radiation,
a list of detector pixels of detector modules that are marked as defective,
an expected influence of an orientation of a collimator, in particular a tilted collimator, on the capture of radiation signals by the detector modules,
a dependence of a detector response of the detector modules on thermal influencing variables.

A response of detector pixels of the detector modules to radiation without an examination object can, for example, be a response of the detector pixels to radiation from air scans. In other words, preferably defined X-rays can be directed at the detector modules or at individual detector modules and it is possible to capture the signal which the detector modules record on this basis. The X-rays can in particular be defined in terms of their intensity and/or frequency. For example, a defined X-ray spectrum with a defined intensity can be directed at the detector modules. The capture of the detector response to radiation can be a good indication of how the respective detector modules are functioning, wherein a complete measurement does not yet have to be performed. Hence, the detector response can be captured at a particularly early stage in the setting up of the X-ray detector or the entire X-ray imaging system. Nevertheless, the method according to one or more example embodiments of the invention can enable a relatively reliable statement about the performance of the entire X-ray detector to be made at an early stage with the aid of the responses of the detector modules.

It can happen that detector modules exhibit variable noise behavior over time. This can also influence the subsequent performance of the X-ray detector. Capturing the temporal noise behavior enables this temporal variability to be also taken into account in the context of the method according to one or more example embodiments of the invention.

Detector modules can react differently to prolonged exposure to radiation. In particular, for example, signal instabilities can occur during capture by the detector modules or by individual detector modules in the case of prolonged high-dose incoming X-rays. Radiation-induced signal instabilities can, for example, be represented by a temporal profile of a measurement signal through a respective detector module, in particular in the case of high-dose radiation. Accordingly different profiles of a measurement signal can represent different degrees of radiation-induced signal instability. For example, a uniform, in particular high-dose, radiation signal can be recorded and deviations from a constant profile represent signal instability.

It has been shown that individual defective detector pixels can have varying degrees of influence on the quality of the X-ray detector. For example, in some cases, an X-ray detector can still function well, even if individual detector pixels are defective. However, the image quality of the X-ray detector can vary depending on the relative position of the defective detector pixels, in particular in relation to one another, and the number of defective detector pixels. The list of detector pixels that are marked as defective can in each case comprise a measure of the severity and/or type of defect. For example, a detector pixel can be marked as defective if a signal response within a defined limit is significantly different than the signal response of other detector pixels in the vicinity. A measure of the severity of the defect can, for example, be the relative deviation of the signal response from an average of signal responses of surrounding detector pixels. It has been shown that the method according to one or more example embodiments of the invention can be used to make a good estimation of the influence of individual defective detector pixels on the overall image quality.

A tilted collimator can, for example, lead to additional scattered radiation influences. For example, shifting the focus of the X-ray tube can cause the collimator to be mapped as a shadow. It can be helpful to estimate scattered radiation influences and other effects of a tilted collimator of the individual detector modules. The collimator is in particular tilted if is not exactly aligned with the focus of the X-ray source, in particular the X-ray tube. When mounting the collimator on the detector module, for example, by gluing and/or screwing, and when screwing the detector module into the detector mechanics, there can typically be tolerances which ensure that the alignment is not exact. If a collimator is positioned unfavorably, the reaction to scattered radiation can be changed. Depending on the projection and signal, this can, for example, lead to differences in brightness in a scan, which can result in artifacts in the image during image reconstruction.

Detector modules can react differently to thermal influences. A thermal influencing variable can, for example, be a temperature and/or a condition that influences the temperature. A thermal influencing variable can, for example, be the temperature of the respective detector module and/or the environment of the respective detector module. A thermal influencing variable can, for example, be a rotational speed of a fan for cooling the X-ray detector and/or the respective detector module. A thermal influencing variable can, for example, be a dose of the incoming X-rays. A large amount of incoming X-rays can cause an increase in the temperature of the detector module. It has been shown that different reactions of different detector modules to thermal influences or to thermal influencing variables can have an effect on the image quality of the X-ray detector. Advantageously, the method according to one or more example embodiments of the invention enables the influence on image quality to be predicted very accurately by using corresponding characterization data relating to the dependence of a detector response of the detector modules on thermal influencing variables.

According to one embodiment, the trained algorithm comprises trained generative artificial intelligence. In particular, the generative artificial intelligence can comprise a diffusion model with at least one denoising block with which the synthetic image data is generated. A diffusion model is generally a generative probability model that can be used to generate new data, in particular image data. A diffusion model is typically based on adding noise to training data, in particular image data for training, during training and then removing it again. The noise can, for example, be Gaussian noise. However, other types of noise are also conceivable. After training, the diffusion model can be used to generate new synthetic data, in particular synthetic image data, from random noise. During the training of the diffusion model, a reference image is rendered noisy in a forward process or diffusion process by successively adding noise to the reference image. This successively renders the reference image noisy. Typically, the result of this diffusion process is a completely noisy distribution (corresponding to a completely noisy image). After the diffusion process, an artificial neural network is trained in an inverse process to remove noise step-by-step in order to generate denoised image data corresponding to the reference image, so that the addition of noise from the diffusion process is inverted. The inverse process or the training of the inverse process takes place step-by-step, in particular by training the neural network to invert the respective diffusion step for each diffusion step. At the end, the individual steps can be concatenated so that noise can be removed from the images. Hence, the neural network is trained to generate image data from noise. During training, this inverse process is approximated by adapting the trainable parameters of the neural network. The inverse process can comprise a denoising block or a plurality of denoising blocks. A denoising block can in particular be adapted to perform the denoising. Optionally, a plurality of denoising blocks can be provided. The denoising blocks can in particular be adapted to be applied one after the other to data to be denoised. This may possibly produce an even better result. The one or more denoising blocks can preferably in each case comprise an artificial neural network, in particular a convolutional neural network. For example, the one or more denoising blocks can be based on a U-Net structure. The concept of a U-Net is in particular described in Ronneberger O, Fischer P, Brox T (2015). “U-Net: Convolutional Networks for Biomedical Image Segmentation”. arXiv: 1505.04597 [cs. CV] and can be applied analogously in the context of the present invention. For example, it can be provided that the resolution in the U-Net is halved until a low final resolution, for example 2×2, is achieved. For example, two ResNet blocks can be provided for each resolution, which in particular contain attention heads. This concept can in particular be token-based, wherein, for example, 32 dimensions can be provided for each token. A ResNet (“residual neural network”) is in particular a deep learning-based model in which the weight layers learn residual functions with reference to the inputs of the layers. Reference is generally also made to “layers” of the model or a neural network.

However, in principle, other structures of artificial neural networks are also conceivable for a denoising block. The inverse process can be linked to a condition as an additional input parameter. This link to a condition can be referred to as conditioning. In the context of the present invention, the condition can in particular comprise the characterization data as an additional input parameter. The condition can, for example, be implemented via an embedding function and/or via a cross-attention mechanism. Hence, training data can comprise real image data and assigned characterization data in each case so that a set of training data can in particular comprise training pairs of real image data and characterization data. Hence, the properties of the detector, which was characterized on the basis of a plurality of scans or measurement results from a test process, can be included as training data in the generation of the synthetic image data.

Advantageously, a very large number of noisy images can be generated from a set of training images. Hence, the artificial neural network is able to learn very precisely from which image domain it should generate samples. It has been shown that a diffusion model, in particular with self-supervised learning, enables sufficient understanding of real image data of the X-ray detector to be developed in order to reliably generate synthetic image data of good quality.

For example, the architecture of the diffusion model can be based on decoder-only transformers, in particular with a cross-attention mechanism to conditioning data. An attention head can, for example, be substantially provided in the conventional way. A feed-forward network at the end of the block can consist of a hidden layer and in particular have a ReLU (rectified linear unit) or ELU (exponential linear unit) as an activation function. For example, a layer normalization operation or RMSNorm (root mean square layer normalization) operation can be used to normalize the values. Overall, a large number of such blocks can be arranged, in particular in a U-Net form, in order to generate synthetic images.

The diffusion model can comprise at least one latent space, an encoder for transferring image data from an image domain into the at least one latent space, a decoder for transferring data from the at least one latent space into the image domain, an embedding function for embedding the characterization data into the at least one latent space, and at least one denoising block. The at least one denoising block can preferably be provided in the at least one latent space. The latent space can also be referred to as an embedding space. In particular, a latent space of the image domain and a latent space of the characterization data can be provided. The latent space of the image domain is preferably a continuous representation, in particular embedding, with a lower dimension than the image domain. The latent space can hence be regarded as a smaller spatial representation of the image data compared to the representation of the image domain. The reduced complexity, corresponding to the lower dimension, enables efficient generation of image data with a diffusion model. Data can be transferred from the image domain into the latent space, in particular into the latent space of the image domain, and vice versa by the encoder or the decoder. Image data can be summarized in the form of coordinates in the latent space, in particular in the latent space of the image domain. Conditioning with the characterization data enables a latent space of the characterization data with regard to the detector properties to be learned during training. The encoder is embodied and/or can be trained to map image data into the latent space, in particular the latent space of the image domain. The decoder is embodied and/or can be trained to generate image data, namely in particular synthetic image data, from the coordinates of the latent space. For example, a ResNet structure can be provided for the encoder and/or the decoder. The ResNet structure of the encoder can be embodied in such a way that it downsamples input data to a lower resolution. Downsampling can preferably be provided in a plurality of blocks. In particular, the ResNet structure of the encoder can be designed to embed image data with a higher resolution value to a lower resolution value. For example, the ResNet structure can be designed to embed image data with 512×512×1 pixels to 32×32×1-values. A downsampling step can, for example, comprise two ResNet layers, in particular a convolutional layer, for example, with a kernel size 3, in which the channels are doubled step-by-step. In other words, if the resolution is halved, the number of channels can be doubled. For example, with a resolution of 32 and 16, a self-attention block with 8 or 16 dimensions can be installed. At the end of the encoder chain, the output can be normalized with GroupNormalization and processed with a further Conv2D layer (for example, kernel size 3) to the channels corresponding to the output resolution, for example 32×32×16 channels. GroupNormalization is known in the prior art; in particular it divides channels into groups and the average and variance are calculated for normalization within each group. The decoder can be constructed in the same way as the encoder, with the direction inverted, in particular by using corresponding upsampling instead of downsampling. The embedding function can be embodied and/or trained to extract detector properties from the characterization data and represent them in the latent space, in particular the latent space of the characterization data. For example, the characterization data can be represented as a vector in the latent space. Different detector modules can, for example, be encoded as numbers. Additionally or alternatively, image data can be represented as a vector in the latent space. Accordingly, noisy image data can be provided as randomly selected latent (noise) vector image information. At least one denoising block can be used to generate a denoised latent vector from a latent noise vector. For example, a cross-attention mechanism can be used to take embedded characterization data into account when generating the denoised latent vector. The cross-attention mechanism can, for example, be based on transformer models. It can preferably be provided that a plurality of denoising blocks is executed in succession, in particular until noise has been sufficiently removed.

According to one embodiment, the generative network (in particular comprising the at least one latent space, the encoder, the decoder, the embedding function and at least one denoising block as components) can have been trained as a whole with a set of training data. The set of training data can in each case comprise pairs of mutually assigned image and characterization data. In particular, the components of the generative network can have been optimized simultaneously.

According to one embodiment, the diffusion model comprises a latent space, an encoder for transferring image data from an image domain into the latent space, a decoder for transferring data from the latent space into the image domain, and an embedding function for embedding the characterization data into the latent space, and at least one denoising block, wherein the generative artificial intelligence is trained in such a way that the encoder, decoder and embedding function were first trained separately, wherein subsequently the at least one denoising block was trained with the aid of the trained encoder, decoder and embedding function. Accordingly, the encoder, decoder and embedding function can have been pretrained before the at least one denoising block was trained with the aid of the pretrained components. The encoder, decoder and/or embedding function can have been trained by self-supervised learning. An auto-encoder structure can be provided to train the encoder and decoder simultaneously so that the image data can be reconstructed once again. Embedding of the characterization data can be trained in a similar way. For example, individual parts of the data can be masked out and the respective network is requested to produce the corresponding data. Accordingly, each of the components can learn to extract the essential information from the data in each case. It can be provided that the latent spaces of the components are optimized for structural similarity, for example, cosine similarity. In particular, vectors generated by the individual components can be optimized in such a way that the vectors of the different components have similar angles to one another (in particular cosine similarity). The similarity can, for example, be optimized with the aid of a loss function. For example, contrastive training, in particular self-supervised contrastive training, can be provided. The loss function can, for example, be a symmetrized cross entropy loss function. Such training can, for example, be provided analogously to that described in Radford, A., “Learning Transferable Visual Models from Natural Language Supervision, 2021. doi: 10.48550/arXiv.2103.00020. It has been shown that such pretraining of individual components can lead to better results as each individual component itself can already be optimized and individual problems of the components can be solved in an improved way. In particular, it has been shown that hence less training data is required to achieve good results overall. For example, it can be possible to train a neural network with a maximum of around 10,000 images. For example, division into training and test data of around 80 to 20 can be provided in order to achieve good results. The training can, for example, be performed using 5-fold cross-validation.

According to one embodiment, the synthetic image data corresponds to synthetic phantom images, in particular synthetic water phantom images. Water phantom images are in particular images that have been recorded with an X-ray imaging system, in particular a computed tomography system, wherein a water phantom has been used as the examination object. Typically, a water phantom is a container filled with water, for example a Plexiglass container filled with distilled water. The water phantom can be used as a substitute for real living tissue. Advantageously, on the one hand, water phantom images can be a good measure of the image quality of the X-ray detector and, on the other hand, large amounts of training data can be generated relatively easily in order to train the algorithm. In particular, fewer radiation protection regulations need to be taken into account when recording water phantom images than would be the case, for example, with live examination objects. For example, pairs of characterization data and water phantom images, which in each case are assigned to an X-ray detector with an arrangement of detector modules, can be used to train the algorithm. Herein, corresponding characterization data can be assigned to each detector module of the X-ray detector.

According to one embodiment, the method comprises the following further steps:
receiving real image data measured by the X-ray detector and corresponding in particular to the synthetic image data;
optionally registering the real image data with the synthetic image data,
retraining the trained algorithm with the real image data as training data.

Optionally, retraining with the real image data can also be carried out on the basis of the characterization data. The real image data and the associated characterization data can be archived for the purpose of retraining. The real image data can be used for retraining, new training and/or fine-tuning the algorithm or another trainable algorithm. Hence, advantageously, an increasingly accurate image of the relevant influencing variables for the image quality can be obtained. For example, it can be provided that the additional training data obtained in this way is added to the existing training data. Retraining can be performed in the same way as the initial training.

According to one or more example embodiments of the invention is a method for quality control in the manufacture of an X-ray detector for an X-ray imaging system, in particular for a computed tomography system, with a plurality of detector modules, wherein the method comprises the following steps:
recording and/or creating characterization data of the detector modules of the X-ray detector, wherein at least part of the characterization data is recorded by measurements with the detector modules without an examination object;
executing the method according to one of the preceding claims with the recorded and/or created characterization data;
analyzing the synthetic image data and, based on the synthetic image data, estimating the quality of the X-ray detector.

The characterization data can, for example, be created with the aid of an X-ray source for generating X-rays. The X-ray source can, for example, comprise an X-ray tube. The characterization data can optionally be created with the aid of a sensor, in particular a temperature sensor. The quality of the X-ray detector can, for example, be estimated based on the evaluation of image artifacts and/or an assessment of whether image artifacts are present and/or the extent to which image artifacts are present. Image artifacts can, for example, be ring artifacts or streak artifacts. The extent of the image artifacts can, for example, be evaluated on the basis of the intensity of existing image artifacts and/or on the basis of a frequency of existing image artifacts. Optionally, additionally or alternatively, an extent of the image artifacts can be evaluated on the basis of a position of the image artifacts on the image data. The quality of the X-ray detector can be estimated automatically, for example, with the aid of an evaluation algorithm. The evaluation algorithm can, for example, be executed and/or provided on a computer unit. The computer unit can, for example, be part of a computer and/or part of the X-ray imaging system. The evaluation algorithm can, for example, be part of a computer program, in particular as described herein. Optionally, the estimation of the quality can be provided by a user, for example, by optical analysis of the synthetic image data.

According to one embodiment, the characterization data can be assigned to an arrangement of the detector modules in the X-ray detector. Herein, a further step can be provided: virtual or actual assembly of the detector modules in the X-ray detector according to the arrangement or an arrangement of the detector modules in the X-ray detector. This step can, for example, be provided before or after the recording and/or creation of the characterization data.

According to one embodiment, a plurality of sets of characterization data is recorded and/or created, wherein the individual sets of characterization data in each case correspond to different arrangements of the detector modules and/or different combinations of detector modules, wherein synthetic image data is generated and analyzed for each of the sets of characterization data. Hence, advantageously, the most suitable arrangement can be found from large number of possible arrangements. In particular, it has been shown that it may be sufficient to create characterization data for each individual detector module, whereby different arrangements of detector modules and/or different combinations of detector modules can then be analyzed. The method can optionally comprise the further step: selecting an arrangement and/or combination of detector modules based on the analysis of the synthetic image data, in particular such that the selection is performed based on the estimation of the image quality in dependence on the arrangement and/or combination of detector modules. For example, if a module exchange is planned, it can already be estimated before the module exchange which of a plurality of available detector modules is suitable.

One or more example embodiments of the invention is a computer-implemented method for training a trainable algorithm comprising:
receiving input training data in the form of characterization data of an X-ray detector for an X-ray imaging system, in particular for a computed tomography system;
receiving output training data, in particular in the form of real image data recorded with the X-ray detector, wherein the output training data is related to the input training data;
training a trainable algorithm based on the input training data and the output training data, wherein the trainable algorithm is in particular based on machine learning;
providing the trained algorithm.

The weights of the trainable algorithm can initially be initialized randomly, for example. To generate the training data, characterization data of an X-ray detector with an arrangement of detector modules can, for example, be created then real image data can be generated with the finished X-ray detector. This characterization data can in particular be related to the respective real image data. The input training data can form a set of training data together with the output training data. Optionally, if such data is present, a database of existing characterization data and associated real image data can be used. The training data can, for example, be designed as a vector and/or matrix. For example, image data can be provided in a resolution of 512×512 to 16384×16384. The image data can, for example, be provided in a HU scale (Hounsfield scale).

For example, it can be provided that the number of real image data and characterization data used for training is based on a number of investigated set-up detectors in the order of 50. Typically, a large number of image data and associated characterization data can be obtained from each set-up detector. This typically enables sufficient information to be collected for different artifacts. Alternatively or additionally, training data can be provided in the form of synthetic data. The synthetic data can, for example, be provided by modifying characterization data and image data using models. Synthetic training data can in particular be provided for pretraining. Training parameters can, for example, be determined and optimized as part of hyperparameter optimization, for example via grid search. In some cases, it can be provided that training data is downscaled, in particular if it turns out in individual cases that this can achieve better training results. For example, image data with a resolution of 512×512 pixels can be scaled down to 256×256 pixels.

According to one embodiment, the trainable algorithm comprises trainable generative artificial intelligence, in particular as described herein. Preferably, the trainable algorithm comprises a diffusion model. In particular, the generative artificial intelligence can comprise a diffusion model with at least one denoising block with which synthetic image data is generated and with a diffusion process. Advantageously, virtually unlimited noisy images or image data can be generated from a set of training images. Hence, the algorithm can learn very precisely from which image domain it should generate samples or synthetic image data.

According to one embodiment, the trainable algorithm, in particular comprising generative artificial intelligence, is trained as a whole with a set of training data. The algorithm trained as a whole can in particular comprise at least one latent space, an encoder, a decoder, an embedding function and at least one denoising block. In particular, it can be provided that the components are trained simultaneously as part of the training.

According to one embodiment, the diffusion model comprises a latent space, an encoder for transferring image data from an image domain into the latent space, a decoder for transferring data from the latent space into the image domain, and an embedding function for embedding the characterization data into the latent space, and at least one denoising block, wherein the generative artificial intelligence is trained in such a way that the encoder, decoder and embedding function are first trained separately, wherein subsequently the at least one denoising block is trained with the aid of the trained encoder, decoder and embedding function. The training can in each case be based on self-supervised learning. Individual parts of the training data can be masked out and predicted by the respective component.

One or more example embodiments of the invention is a computer program product or a storage medium, in particular a non-volatile storage medium comprising instructions which, when executed by a computer, cause the computer to execute the steps of a proposed method as described herein, in particular a computer-implemented method for supporting the evaluation of characterization data of an X-ray detector for an X-ray imaging system, a method for quality control in the manufacture of an X-ray detector for an X-ray imaging system and/or a method for training a trainable algorithm. All advantages and features of the proposed method can be transferred analogously to the computer program product or the storage medium and vice versa. The computer program product can, for example, be stored on a computer-readable storage medium, in particular a non-volatile storage medium. The storage medium can, for example, be a hard disk, an SSD, a flash memory, an online server, etc.

One or more example embodiments of the invention is a system comprising an interface for receiving characterization data from detector modules of an X-ray detector, and a computer unit which is connected to the interface and configured to execute a proposed method as described herein. All advantages and features of the method and the computer program product or the storage medium can be transferred analogously to the system and vice versa.

All embodiments described herein can be combined with one another, unless explicitly stated otherwise.

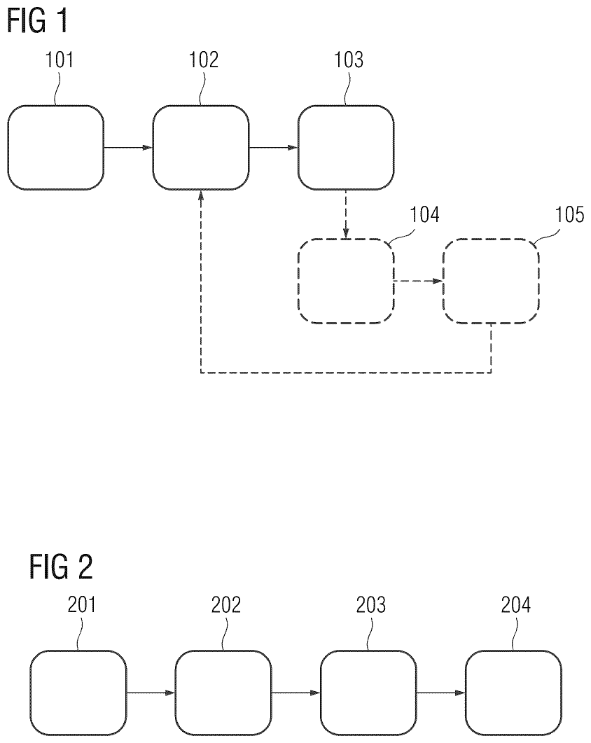

FIG. 1 shows a flowchart for a computer-implemented method for supporting the evaluation of characterization data 31 of an X-ray detector for an X-ray imaging system according to one embodiment of the invention. The X-ray imaging system can in particular be a computed tomography system. The X-ray detector comprises a plurality of detector modules. In a first step 101 of the method, characterization data 31 of the detector modules of the X-ray detector is received. At least part of the characterization data 31 is based on measurement data of the detector modules recorded without an examination object. In particular, characterization data 31 can be assigned to an arrangement of the detector modules in the X-ray detector. In other words, characterization data 31 can comprise information that defines the arrangement in which the detector modules are arranged and/or provided in the X-ray detector. For example, the characterization data 31 can comprise a response of detector pixels of the detector modules to radiation without an examination object. Additionally or alternatively, the characterization data 31 can, for example, comprise temporal noise behavior of individual detector pixels of the detector modules. Additionally or alternatively, the characterization data 31 can, for example, comprise signal instabilities of the detector modules induced by incoming radiation. comprise a list of detector pixels of the detector modules that are marked as defective. Additionally or alternatively, the characterization data 31 can, for example, comprise an expected influence of an orientation of a collimator, in particular a tilted collimator, on the capturing of radiation signals by the detector modules. Additionally or alternatively, the characterization data 31 can, for example, comprise a dependence of a detector response of the detector modules on thermal influencing variables. In a further step 102, a trained algorithm is applied to the characterization data 31. The trained algorithm can in particular comprise trained generative artificial intelligence. The trained generative artificial intelligence can, for example, be or comprise a diffusion model. The output generated by the trained algorithm is synthetic image data 14 that simulates image data 13 of an X-ray imaging system recorded with the X-ray detector. The synthetic image data 14 can comprise raw data corresponding to raw data recorded with the X-ray detector. The synthetic image data 14 can comprise images corresponding to reconstructed images recorded with the X-ray detector. The synthetic image data 14 can, for example, correspond to synthetic phantom images, in particular synthetic water phantom images. In a further step 103, the synthetic image data 14 is provided. Provision can, for example, comprise storing the synthetic image data 14, forwarding via a network to another system and/or another data memory, and/or outputting to a user interface. Optionally, this method, or further embodiments of methods described herein, can comprise further steps 104, 105 for retraining the trained algorithm. For this purpose, in a step 104, real image data 13 measured by the X-ray detector that was subsequently set up and integrated into an X-ray imaging system is received. In particular, the real image data 13 corresponds to the synthetic image data 14. This can in particular be understood to mean that the real image data 13 represents or is intended to represent the same subject matter as the synthetic image data 14 and/or was generated under measurement conditions that correspond to the measurement conditions simulated for the synthetic image data 14. This is because the measurement conditions simulated for the synthetic image data 14 can, for example, correspond to the measurement conditions of the real image data 13 in which the real image data 13 was recorded under the measurement conditions under which the training data with which the trained algorithm was originally trained was also recorded. The real image data 13 can optionally be registered with the synthetic image data 14. In a further step 105, the trained algorithm is retrained with the real image data 13 as training data. For further applications of the method the retrained algorithm, i.e. possibly adapted algorithm, can in particular be used.

FIG. 2 shows a flowchart for a computer-implemented method for training a trainable algorithm according to one embodiment of the invention. In a first step 201, input training data in the form of characterization data 31 of an X-ray detector for an X-ray imaging system, in particular for a computed tomography system, is received. In a further step 202, output training data in the form of real image data 13 recorded with the X-ray detector is received. Preferably, the output training data is related to the input training data or is linked thereto. In particular, the output training data and some training data are linked as training pairs. In a further step 203, a trainable algorithm is trained based on the input training data and the output training data. The trainable algorithm can in particular be based on machine learning. For example, the trainable algorithm can be a diffusion model. In a further step 204, the trained algorithm that is now trained is provided.

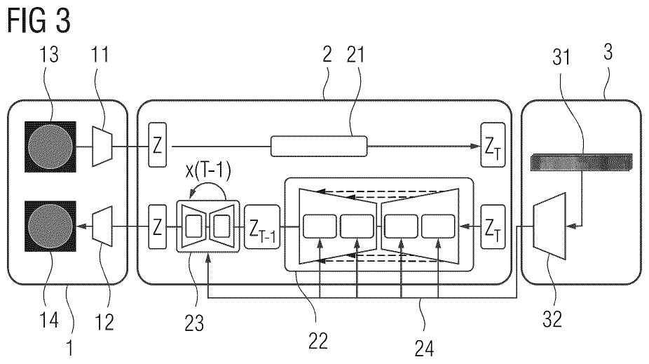

FIG. 3 shows a schematic diagram of the structure of a trained or trainable algorithm according to one embodiment of the invention. In this example, the algorithm comprises generative artificial intelligence based on a diffusion model. The diffusion model can, for example, be implemented and trained analogously as described in Robin Rombach, A. Blattmann, Dominik Lorenz, Patrick Esser, and Bjorn Ommer: “High-Resolution Image Synthesis with Latent Diffusion Models”, CVPR, 2022. The diffusion model comprises a latent space 2, an encoder 11 for transferring image data from an image domain 1 into the latent space 2, a decoder 12 for transferring data from the latent space 2 into the image domain 1 and an embedding function 32 for embedding characterization data 31 from a raw data domain 3 into the latent space 2. A denoising block 22, at least one further denoising block 23 and a diffusion process 21 are provided in the latent space 2. The encoder 11 is used to map image data from an image domain 1 into the latent space 2, in particular as coordinates or as a vector. The decoder 12 is used to map coordinates from the latent space 2 into the image domain 1 or to generate image data, in particular images, in the image domain 1 from the coordinates in the latent space 2. In this example, the image data 13, 14 is water phantom images. For example, in each case a ResNet structure can be provided for the encoder and the decoder. The ResNet structure of the encoder can be designed in such a way that it downsamples input data in a plurality of blocks to a lower resolution. For example, the ResNet structure can be designed to embed image data with 512×512×1 pixels to 32×32×1-values. A downsampling step can, for example, comprise two ResNet layers, in particular a convolutional layer with kernel size 3, in which the channels are doubled step-by-step. In other words, if the resolution is halved, the number of channels can be doubled. For example, with a resolution of 32 and 16, a self-attention block with 8 or 16 dimensions can be installed. At the end of the encoder chain, the output can be normalized with GroupNormalization and processed with a further Conv2D layer (kernel size 3) to the 32×32×16 channels, for example. The decoder can be set up in the opposite direction to the encoder by using upsampling instead of downsampling. On the other hand, the embedding function 32 is used to embed the characterization data 31 into the latent space 2. Herein, at least one denoising block 22, 23 is responsible for the generative process for generating the synthetic image data 14. During training, the diffusion process 21 adds noise to the real image data 13 mapped into the latent space 2 via the encoder 11 and then removes it again via the denoising blocks 22, 23. The noise can, for example, be Gaussian noise. In the diffusion process 21, a real image Z mapped in the latent space 2 and which serves as a reference image is rendered noisy by successively adding noise to the reference image until typically a completely noisy distribution ZT corresponding to a completely noisy image is present. After the diffusion process 21, the denoising blocks 22, 23 are trained in an inverse process to remove noise step-by-step so that a denoised distribution Z is generated in order to finally generate denoised synthetic image data 14 corresponding to the real image data 13 via the decoder 12. Herein, the addition of noise from the diffusion process 21 is inverted. After the first denoising block 22, there is initially a partially noisy distribution ZT-1, which is finally converted into the completely denoised distribution Z after at least one further denoising block 23 has been applied. Hence, the denoising blocks 22, 23 are trained to generate image data 14 from noise. During training, this inverse process is approximated by adapting the trainable parameters of the denoising blocks 22, 23. The inverse process in the denoising blocks 22, 23 is linked to a condition as an additional input parameter (conditioning), wherein this condition comprises the characterization data 31 as an additional input parameter which is embedded into the latent space 2 via the embedding function 32 and enters the inverse process via a cross-attention mechanism 24. This cross-attention mechanism 24 can, for example, be implemented as described in Robin Rombach, A. Blattmann, Dominik Lorenz, Patrick Esser, and Bjorn Ommer: “High-Resolution Image Synthesis with Latent Diffusion Models”, CVPR, 2022. Hence, training data can comprise sets of training pairs of real image data 14 and characterization data 31. After training, the diffusion model can be used to generate new synthetic data, in particular synthetic image data 14, from random noise. Starting from a randomly selected latent vector for image information, a new denoised latent vector is generated via the cross-attention mechanism 24 via which the characterization data 31 embedded into the latent space 2 is received. This is executed several times in succession, in particular with the addition of at least one further denoising block 23 until the noise has been sufficiently removed and image data 14, in particular images, can be generated via the decoder 12. In the exemplary embodiment shown, the denoising block 22 and the at least one further denoising block 23 are in each case set up in a U-Net architecture. The U-Net architecture can be set up similarly to the architecture of the encoder 11 and decoder 12. For example, it can be provided that the resolution in the U-Net is halved until a resolution of 2×2 is achieved. Two ResNet blocks can be provided for each resolution which in particular contain attention heads. This concept can in particular be token-based, wherein, for example, 32 dimensions can be provided for each token. However, as an alternative to a U-Net architecture, other architectures are also conceivable which allow the integration of a suitable cross-attention mechanism 24 and which can be trained efficiently. Hence, an input for the decoder 12 can be created in the latent space 2 via the inverse diffusion process (inverse process 25) of the denoising blocks 22, 23 based on the detector characterization context of the characterization data 31 and, finally, image data for the X-ray detector can be predicted in the form of the synthetic image data 14. Through the characterization data 31, properties of the X-ray detector or the detector modules of the X-ray detector flow into the generation process for generating synthetic image data 14. In the example shown here, the characterization data 31 comprises an air shot of the entire X-ray detector (foremost image of the characterization data 31 shown), wherein each strip shown in the image is in each case assigned to a detector module. In addition, further characterization data 31, such as, for example, detector measurement data of the detector modules with a shifted focus, is provided as input. While the characterization data 31 is illustrated here as images, it can in particular be provided that the characterization data 31 is actually entered as raw data or numerical values.

In this embodiment, the method according to one or more example embodiments of the invention is based on the fact that sufficient understanding of the image data of an X-ray detector can be developed by the diffusion process 21 through self-supervised learning, which is summarized in the form of coordinates in the latent space 2. At the same time, a latent space 2 of the detector properties is learned through conditioning with the characterization data 31. The structure of the algorithm allows the characterization data 31 and the image data 13, 14 to be correlated. This makes use of the fact that the detector properties characterized by the characterization data 31 can be decisive for any artifacts observed in the images.

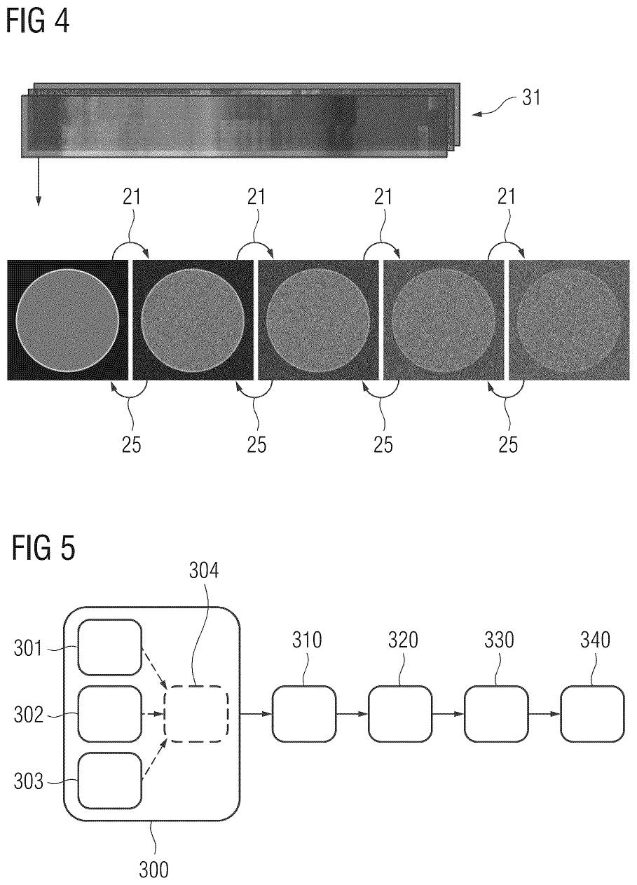

FIG. 4 shows schematically the mode of operation of the diffusion model shown in FIG. 3. During training, Gaussian noise is successively added to a water phantom image in a diffusion process 21 (forward process). In the examples shown here, the water phantom image is largely artifact-free. In an inverse process 25, the noise is successively removed again by the denoising blocks 22, 23. Herein, the inverse process is conditioned by the characterization data 31. After training, the characterization data 31 can be used together with noise (for example, randomly generated noise) to generate synthetic image data 14 in the inverse process 25.

FIG. 5 shows a flowchart for a computer-implemented method for training a trainable algorithm based on a diffusion model according to one embodiment of the invention. In a first step 300, pretraining steps 301-303 are first performed by training the encoder 11 in a step 301, the decoder 12 in a further step 302 and the embedding function 32 in yet a further step 303. The pretraining of the individual components can in particular be realized by unsupervised learning, wherein individual parts of the data are masked out in each case and are to be predicted by the network. Herein, each of the components 11, 12, 32, which are provided for the transition into the latent space 2 or out of the latent space 2, learns on its own to extract the essential information from the data. These pretraining steps can, for example, take place in parallel or (optionally partially) one after the other. Optionally, after training the individual components, the maps can be optimized for structural similarity in a further step 304, for example via their cosine similarity. Subsequently, at least one denoising block 22, 23 of the diffusion model is trained with the aid of the trained encoder 11, decoder 12 and embedding function 32 in the further steps 310-340. For this purpose, input training data in the form of characterization data 31 of an X-ray detector for an X-ray imaging system, in particular for a computed tomography system, is received in a step 310. In a further step 320, output training data in the form of real image data 13 recorded by the X-ray detector is received. In particular, the output training data and some training data can be linked as training pairs. In a further step 330, a diffusion model is trained as a whole based on the input training data and the output training data. In a further step 340, the now fully trained diffusion model is provided.

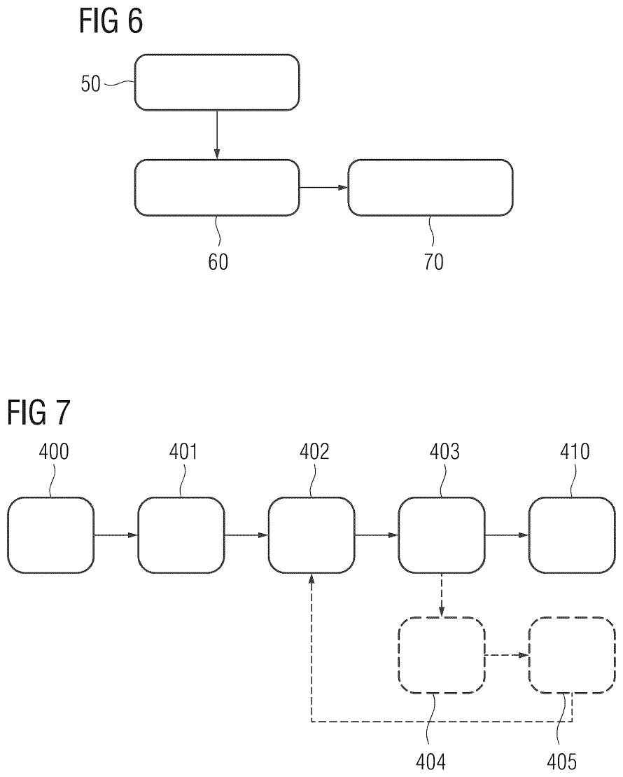

FIG. 6 shows a schematic structure of a system according to one embodiment of the invention. The system comprises an interface 50 for receiving characterization data 31 from detector modules of an X-ray detector and a computer unit 60 connected to the interface 50 and configured to execute the method as described herein. To output the synthetic image data 14, a further interface 70 is provided, which is connected to the computer unit 60. Optionally, the interface 50 and the further interface 70 can be integrated in a common interface.

FIG. 7 shows a method for quality control in the manufacture of an X-ray detector for an X-ray imaging system, in particular for a computed tomography system, with a plurality of detector modules, according to one embodiment of the invention. In a first step 400, characterization data 31 of the detector modules of the X-ray detector is recorded and/or created, wherein at least part of the characterization data 31 is recorded by measurements with the detector modules without an examination object, wherein the characterization data 31 is in particular assigned to an arrangement of the detector modules in the X-ray detector. Optionally, a plurality of sets of characterization data 31 can be recorded and/or created, wherein the individual sets of characterization data 31 in each case correspond to different arrangements of the detector modules and/or different combinations of detector modules. In further steps 401-403, the steps 401-403 of a method as described for example with reference to FIG. 1, are executed with the recorded and/or created characterization data 31. Herein, the steps 401-403 shown here correspond in particular to the steps 101-103 of the method shown in FIG. 1. If a plurality of sets of characterization data 31 have been recorded and/or created, synthetic image data 14 can be generated for each of the sets of characterization data 31. In a further step 410, the synthetic image data 14 is analyzed and, based on the synthetic image data 14, the quality of the X-ray detector is estimated. If a plurality of sets of characterization data 31 is recorded and/or created and synthetic image data 14 has been generated for each of the sets of characterization data 31, the synthetic image data 14 can be analyzed for each of the sets of characterization data 31 in each case. Here, once again, the retraining steps 404-405 can optionally be provided, analogously to retraining steps 104-105 in FIG. 1.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various s information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A computer-implemented method for supporting an evaluation of characterization data of an X-ray detector for an X-ray imaging system, the method comprising:
receiving the characterization data for detector modules of the X-ray detector, wherein at least part of the characterization data is based on measurement data of the detector modules recorded without an examination object;
applying a trained algorithm to the characterization data to generate synthetic image data simulating image data of the X-ray imaging system recorded with the X-ray detector; and
providing the synthetic image data.

2. The method of claim 1, wherein the characterization data is assigned to an arrangement of the detector modules in the X-ray detector.

3. The method of claim 1, wherein the characterization data comprises one or more of:
a response of detector pixels of the detector modules to radiation without an examination object,
temporal noise behavior of individual detector pixels of the detector modules,
signal instabilities of the detector modules induced by incoming radiation,
a list of detector pixels of the detector modules marked as defective,
an expected influence of an orientation of a collimator on a capture of radiation signals by the detector modules, or
a dependence of a detector response of the detector modules on thermal influencing variables.

4. The method of claim 1, wherein the trained algorithm comprises trained generative artificial intelligence.

5. The method of claim 15, wherein the diffusion model comprises:
at least one latent space,
an encoder configured to transfer image data from an image domain into the at least one latent space,
a decoder configured to transfer data from the at least one latent space into the image domain,
an embedding function configured to embed the characterization data into the at least one latent space, and
at least one denoising block, wherein the generative artificial intelligence is trained such the encoder, decoder and embedding function have been trained separately, and the at least one denoising block has subsequently been trained with the trained encoder, the trained decoder and the trained embedding function.

6. The method of claim 1, wherein the synthetic image data corresponds to synthetic phantom images.

7. The method of claim 1, further comprising:
receiving real image data measured by the X-ray detector, the real image data corresponding to the synthetic image data;
optionally registering the real image data with the synthetic image data; and
retraining the trained algorithm with the real image data as training data.

8. A method for quality control, the method comprising:
executing the method of claim 1; and
analyzing the synthetic image data and estimating the quality of the X-ray detector based on the synthetic image data.

9. The method of claim 8, wherein the characterization data is assigned to an arrangement of the detector modules in the X-ray detector.

10. The method of claim 8, wherein
a plurality of sets of characterization data is at least one of recorded or created,
each set of characterization data corresponds to at least one of different arrangements of the detector modules or different combinations of detector modules, and
synthetic image data is generated and analyzed for each of the sets of characterization data.

11. A computer-implemented method for training a trainable algorithm comprising:
receiving input training data, the input training data being characterization data of an X-ray detector for an X-ray imaging system;
receiving output training data, the output training data being real image data recorded with the X-ray detector, the output training data being related to the input training data;
training a trainable algorithm based on the input training data and the output training data, wherein the trainable algorithm is based on machine learning; and
providing the trained algorithm.

12. A non-transitory computer readable medium comprising instructions which, when executed by a computer, cause the computer to perform the method of claim 1.

13. A system comprising:
an interface configured to receive the characterization data from the detector modules of the X-ray detector; and
a computer unit connected to the interface and configured to perform the method of claim 1.

14. The method of claim 1, wherein the X-ray imaging system is a computed tomography system.

15. The method of claim 4, wherein the generative artificial intelligence comprises a diffusion model with at least one denoising block, the synthetic image data being generated using the diffusion model.

16. The method of claim 6, wherein the synthetic phantom images are synthetic water phantom images.

17. The method of claim 2, wherein the characterization data comprises one or more of the following:
a response of detector pixels of the detector modules to radiation without an examination object,
temporal noise behavior of individual detector pixels of the detector modules,
signal instabilities of the detector modules induced by incoming radiation,
a list of detector pixels of the detector modules marked as defective,
an expected influence of an orientation of a collimator on a capture of radiation signals by the detector modules, or
a dependence of a detector response of the detector modules on thermal influencing variables.

18. The method of claim 2, further comprising:
receiving real image data measured by the X-ray detector, the real image data corresponding to the synthetic image data;
optionally registering the real image data with the synthetic image data; and
retraining the trained algorithm with the real image data as training data.
